(19) United States
(12) Patent Application Publication
(10) Pub. No.: US 2009/0270756 A1
(43) Pub. Date: Oct. 29, 2009

(54) DETERMINING PHYSIOLOGICAL CHARACTERISTICS OF ANIMAL

(76) Inventors: Ronald W. Gamache, East Greenbush, NY (US); Sarah Pluta, Scotia, NY (US)

Correspondence Address: HOFFMAN WARNICK LLC, 75 STATE STREET, 14TH FLOOR, ALBANY, NY, 12207, US

(21) Appl. No.: 12/258,509

(22) Filed: Oct. 27, 2008

Related U.S. Application Data
(60) Provisional application No. 61/047,199, filed on Apr. 23, 2008.

(51) Int. Cl. A61B 5/053

(52) U.S. Cl. 600/547

(57) ABSTRACT

A system, method and program product enable determining physiological characteristics of an animal. In one embodiment, the system includes a sensor having an array of electrodes for use in obtaining complex impedance data from a body part of an animal; and a determinater that compares the complex impedance data with an empirical data model to determine a physiological parameter of the animal, the empirical data model including physiological parameter data versus complex impedance data value correspondence of the animal.

### CROSS-REFERENCE TO RELATED APPLICATIONS

This application claims priority to U.S. provisional application No. 61/047,199, which is hereby incorporated by reference.

### BACKGROUND

The present disclosure relates to a method, system and program product for determining physiological characteristics of an animal.

### SUMMARY

A system, method and program product are disclosed that enable determining physiological characteristics of an animal. In one embodiment, the system includes a sensor having an array of electrodes for use in obtaining complex impedance data from a body part of an animal; and a determinater that compares the complex impedance data with an empirical data model to determine a physiological parameter of the animal, the empirical data model including physiological parameter data versus complex impedance data value correspondence of the animal.

A first aspect of the invention provides a system for determining physiological characteristics of an animal, the system comprising: a sensor having an array of electrodes for use in obtaining complex impedance data from a body part of an animal; and a determinater that compares the complex impedance data with an empirical data model to determine a physiological parameter of the animal, the empirical data model including physiological parameter data versus complex impedance data value correspondence of the animal.

A second aspect of the invention provides a method for determining physiological characteristics of an animal, the method comprising: obtaining complex impedance data for an animal; and determining a physiological parameter of the animal based on the complex impedance data.

A third aspect of the invention provides a program product stored on a computer readable medium, which when executed, performs the following: obtaining complex impedance data for an animal; determining a physiological parameter of the animal based on the complex impedance data; and outputting the physiological parameter.

A fourth aspect of the invention provides a system for determining physiological characteristics of an animal, the system comprising: an obtainer for obtaining complex impedance data for an animal; and a determinater for determining a physiological parameter of the animal based on the complex impedance data.

The illustrative aspects of the present invention are designed to solve the problems herein described and/or other problems not discussed.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a block diagram of an illustrative environment and computer infrastructure for implementing one embodiment of the invention.

FIG. 2 shows a flow diagram of embodiments of processing complex impedance data using the system of FIG. 1.

FIGS. 3A & 3B show an underside and a side view, respectively, of one embodiment of the sensor of FIG. 1.

FIG. 4 shows a body part of an animal in contact with the sensor of FIG. 1.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION

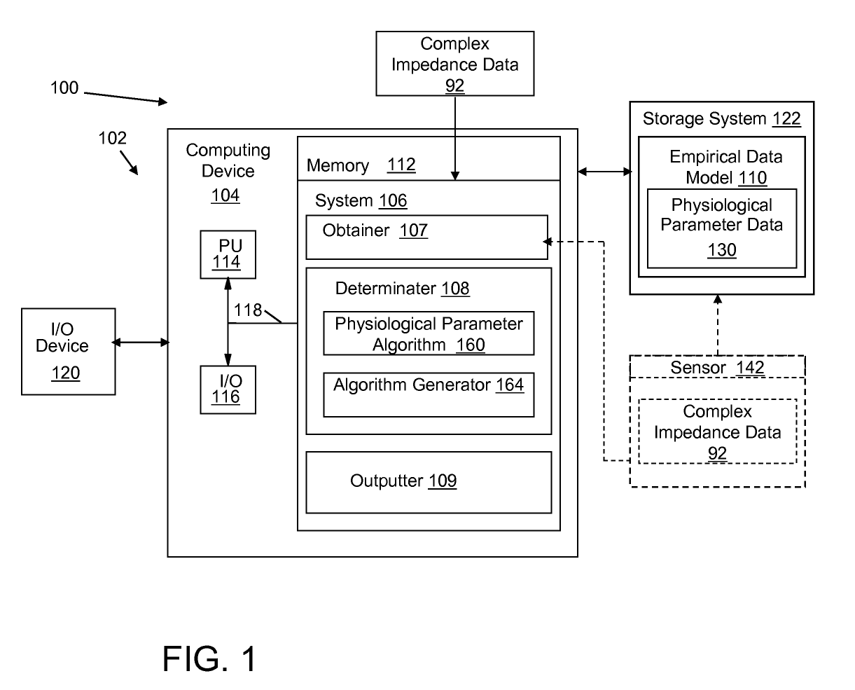

Turning to the drawings, FIG. 1 shows illustrative environment 100 for determining physiological characteristics of an animal. To this extent, environment 100 includes a computer infrastructure 102 that can perform the various processes described herein. In particular, computer infrastructure 102 is shown including a computing device 104 that comprises a system 106, which enables computing device 104 to enable determining physiological characteristics of an animal by performing the steps of the disclosure.

Computing device 104 is shown including a memory 112, a processor unit (PU) 114, an input/output (I/O) interface 116, and a bus 118. Further, computing device 104 is shown in communication with an external I/O device/resource 120 and a storage system 122. In general, processor unit 114 executes computer program code, such as system 106, which is stored in memory 112 and/or storage system 122. While executing computer program code, processor unit 114 can read and/or write data, such as complex impedance data 92, to/from memory 112, storage system 122, and/or I/O interface 116. Bus 118 provides a communications link between each of the components in computing device 104. I/O device 120 can comprise any device that enables a user to interact with computing device 104 or any device that enables computing device 104 to communicate with one or more other computing devices. Input/output devices (including but not limited to keyboards, displays, pointing devices, etc.) can be coupled to the system either directly or through intervening I/O controllers.

In any event, computing device 104 can comprise any general purpose computing article of manufacture capable of executing computer program code installed by a user (e.g., a personal computer, server, handheld device, etc.). However, it is understood that computing device 104 and system 106 are only representative of various possible equivalent computing devices that may perform the various process steps of the invention. To this extent, in other embodiments, computing device 104 can comprise any specific purpose computing article of manufacture comprising hardware and/or computer program code for performing specific functions, any computing article of manufacture that comprises a combination of specific purpose and general purpose hardware/software, or the like. In each case, the program code and/or hardware can be created using standard programming and engineering techniques, respectively.

Similarly, computer infrastructure 102 is only illustrative of various types of computer infrastructures for implementing the invention. For example, in one embodiment, computer infrastructure 102 comprises two or more computing devices (e.g., a server cluster) that communicate over any type of wired and/or wireless communications link, such as a network, a shared memory, or the like, to perform the various process steps of the invention. When the communications link comprises a network, the network can comprise any combination of one or more types of networks (e.g., the Internet, a wide area network, a local area network, a virtual private network, etc.). Regardless, communications between the computing devices may utilize any combination of various types of transmission techniques.

As previously mentioned and discussed further below, system 106 enables computing infrastructure 102 to determine physiological characteristics of an animal. To this extent, system 106 is shown including an obtainer 107, a determinater 108 and an outputter 109. Determinater 108 includes a physiological parameter algorithm 160, which may be generated by an algorithm generator 164. Also shown in FIG. 1 is storage system 122, which may include empirical data model 110. Empirical data model 110 may include value correspondence between physiological parameter data 130 and complex impedance data 92. Optionally, illustrative environment 100 may include sensor 142 (shown in phantom), which may transmit complex impedance data 92 to one or both of storage system 122 and obtainer 107. Operation of each of these functions is discussed further below. However, it is understood that some of the various functions shown in FIG. 1 can be implemented independently, combined, and/or stored in memory for one or more separate computing devices that are included in computer infrastructure 102. Further, it is understood that some of the systems and/or functionality may not be implemented, or additional systems and/or functionality may be included as part of environment 100.

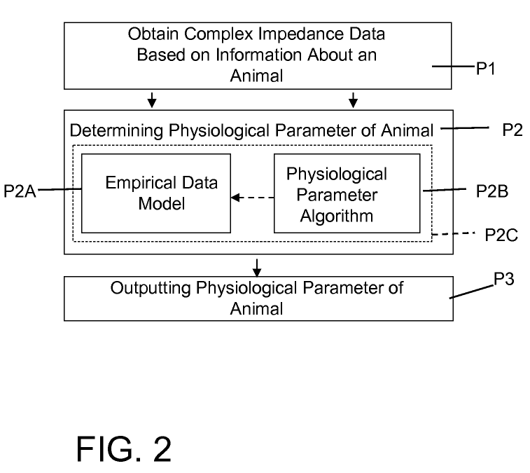

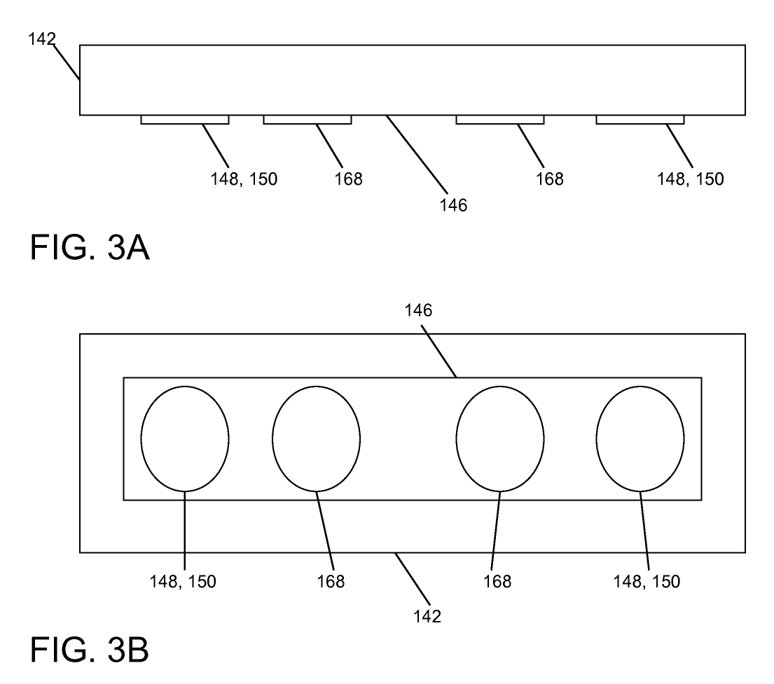

Turning to FIGS. 2-4, and with continuing reference to FIG. 1, embodiments of a method for determining physiological characteristics of an animal will now be described. Complex impedance data 92 may include any form of data gathered by measuring the resistance of a body part of an animal to an electrical signal, such as, for example, an alternating current signal. For example, complex impedance data may include impedance spectral data. Impedance spectral data may be calculated by measuring the resistance of a body part of an animal across a range of frequencies. In process P1, obtainer 107 obtains complex impedance data 92 for an animal. In one embodiment, a sensor 142 having an array of electrodes 146 for use in obtaining complex impedance data 92 from a body part of an animal 220 is used. Turning to FIGS. 3A-3B, an example of a sensor 142 having an array of electrodes 146 is shown. Sensor 142 has array of electrodes 146, which may include, for example, a current transmitting electrode 148, 150, a current sensing electrode 148, 150, and two voltage sensing electrodes 168. Operation of each of these elements is discussed herein. In any case, arrangement of sensor 142 and electrodes 146 are shown merely for illustrative purposes. Current transmitting electrode 148, 150, current sensing electrode 148, 150, and two voltage sensing electrodes 168 may be positioned on sensor 142 in other arrangements than those shown in FIGS. 3A-3B. For example, voltage sensing electrodes 168 may, for example, be positioned between current transmitting electrode 148, 150 and current sensing electrode 148, 150 in a linear arrangement. However, current transmitting electrode 148, 150 and current sensing electrode 148, 150 may, for example, be positioned between the two voltage sensing electrodes 168 in a linear arrangement. Further, sensor 142 and electrodes 146 may, for example, be configured in other arrangements such as circular or arced arrangements. Sensor 142 may contain fewer or greater numbers of electrodes 146 than those shown in FIGS. 3A-3B. Sensor 142 and array of electrodes 146 may be formed of conductive materials including, for example, silver/silver chloride, platinum or carbon. However, sensor 142 and array of electrodes 146 may be formed of other conductive materials now known or later developed.

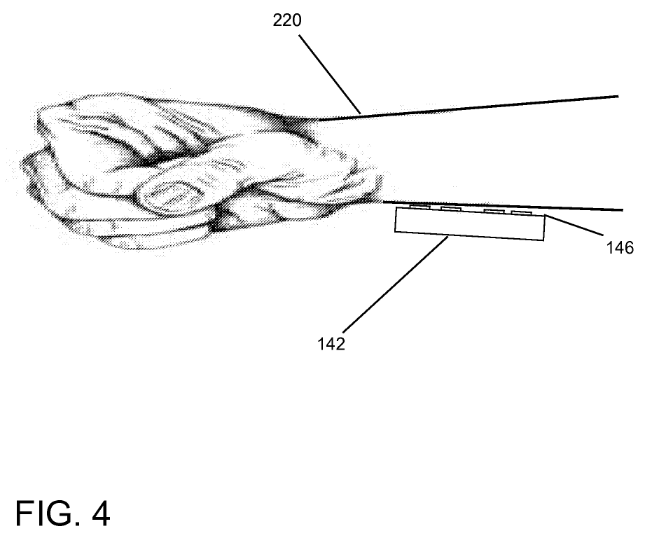

Turning to FIG. 4, and with continuing reference to FIGS. 3A-B, one embodiment of sensor 142 having an array of electrodes 146 is shown in contact with a body part of animal 220. Although a human arm is shown, it is understood that body part of animal 220 could be of any animal or any part of animal. In operation, array of electrodes 146 obtains complex impedance data 92 from the body part of animal 220. Current transmitting electrode 148 and current sensing electrode 150 create an electrical circuit which uses the body part of animal 220 as a conducting medium. Current transmitting electrode 148 may produce a signal which is transmitted through the body part of animal 220, and received by current sensing electrode 150. In one example, the signal transmitted may be an alternating-current signal and may be of a frequency that maximizes extraction of one physiological parameter from the animal. This frequency may range from about 100 Hz to about 10 MHz. When a signal is transmitted through the body part of animal 220, a voltage differential is generated within the body part of animal 220. Voltage sensing electrodes 168 determine this voltage differential within body part of animal 220, and sensor 142 is capable of transmitting this voltage differential as complex impedance data 92 to obtainer 107.

In another embodiment, obtainer 107 obtains complex impedance data 92 for the animal from any source capable of storing and/or transmitting data. For example, complex impedance data 92 may be obtained from a data center, multiple data centers, dispersed or “cloud” data centers, individual data files, or a sensor. These examples are merely illustrative, as obtainer 107 may obtain complex impedance data 92 from any now known or later developed data storage and/or transmission device.

In process P2, determinater 108 determines a physiological parameter of an animal based on complex impedance data 92. This process may occur in several ways. In one embodiment, in process P2A, determinater 108 compares complex impedance data 92 with empirical data model 110. Empirical data model 110 may include physiological parameter data versus complex impedance data value correspondence of an animal. Physiological parameter data versus complex impedance data value correspondence may be specific to a particular animal, or may be generalized for a variety of animals. Physiological parameter data versus complex impedance data value correspondence may be based upon, for example, animal height, weight, sex, age or the like. Empirical data model 110 and physiological parameter data versus complex impedance data value correspondence may be generated from physiological parameter data 130 and complex impedance data 92. Physiological parameter data 130 may be derived from data provided by, for example, physiological testing of animals. Complex impedance data 92 may be derived from, for example, experimentation and/or data collection. In this embodiment, upon receiving complex impedance data 92 from obtainer 107, determinater 108 processes complex impedance data 92 using empirical data model 110 in order to determine a physiological parameter of the animal. The physiological parameter of the animal may include, for example, osmolarity, lactic acid concentration, ionic concentration or glucose concentration. Ionic concentration may include, for example, sodium concentration, chloride concentration, potassium concentration, calcium concentration, bicarbonate concentration and magnesium concentration.

In another embodiment, in process P2B, determinater 108 determines a physiological parameter of the animal using an algorithm 160. Physiological parameter algorithm 160 may be generated by algorithm generator 164. Algorithm generator 164 may use a variety of mathematical analyses in generating physiological parameter algorithm 160. In one case, algorithm generator 164 may use a multivariate analysis including, for example, pattern recognition, principal component analysis, or structure data analysis. Further, in performing structure data analysis, algorithm generator 164 may perform a regression analysis. Algorithm generator 164 may also use ratios, accumulative changes and accumulative differences to generate physiological parameter algorithm 160. Regardless, algorithm generator 164 may use any form of mathematical analysis to generate physiological parameter algorithm 160. In this embodiment, upon receiving complex impedance data 92 from obtainer 107, complex impedance data 92 is processed by physiological parameter algorithm 160 in order to determine a physiological parameter for the animal.

In an optional embodiment, shown in process P2C, both process P2A and process P2B may be combined to determine a physiological parameter for the animal. For example, physiological parameter algorithm 160 and empirical data model 110 may be designed such that complex impedance data 92 processed by physiological parameter algorithm 160 is output to empirical data model 110. In this case, empirical data model 110 may contain corresponding information between one or more physiological parameters and resulting data generated by physiological parameter algorithm 160. In this embodiment, physiological parameter algorithm 160 may process complex impedance data 92 and generate physiological parameter algorithm data that is compatible with empirical data model 110. Determinater 108 may then process the physiological parameter algorithm data using empirical data model 110 in order to determine a physiological parameter of the animal. In an alternate embodiment, a parametric inversion model (not shown) may be used to convert complex impedance data 92 processed by physiological parameter algorithm 160 into parametric inversion model data compatible with empirical data model 110. Parametric inversion model may include, for example, empirical data model to physiological parameter algorithm data value correspondence. This correspondence may be based upon parametric statistics, which may include, for example, a parameterized family of probability distributions. The parameterized family of probability distributions may include an exponential family, a location-scale family, or the like. In this embodiment, physiological parameter algorithm 160 may process complex impedance data 92 and generate physiological parameter algorithm data that is compatible with parametric inversion model. Determinater 108 may process physiological parameter algorithm data using the parametric inversion model to create parametric inversion data compatible with empirical data model 110. The parametric inversion model may compare physiological parameter algorithm data with a probability distribution related to, for example, empirical data about an animal. Using a probability distribution, the parametric inversion model may create parametric inversion model data that is compatible with empirical data model 110. Determinater 108 may then process the parametric inversion data using empirical data model 110 in order to determine a physiological parameter of the animal.

In process P3, outputter 109 outputs the physiological parameter of the animal. Output of the physiological parameter of the animal may be performed by any now known or later developed means. For example, outputter 109 may output the physiological parameter through I/O 116 directly to an I/O device 120, such as a printer, display device, audio device, or the like. Once output, physiological parameter of the animal may be used, for example, in analysis or diagnosis of disease or health conditions. Analysis and diagnosis of the physiological parameter may be performed, for example, with respect to one particular animal, a grouping of animals, or an entire species of animals. Further, analysis and diagnosis of the physiological parameter may be performed in any now known or later developed manner.

As discussed herein, various systems and components are described as “obtaining” data (e.g., obtainer 107). It is understood that the corresponding data can be obtained using any solution. For example, the corresponding system/component can generate and/or be used to generate the data, retrieve the data from one or more data stores (e.g., a database), receive the data from another system/component, and/or the like. When the data is not generated by the particular system/component, it is understood that another system/component can be implemented apart from the system/component shown, which generates the data and provides it to the system/component and/or stores the data for access by the system/component.

While shown and described herein as a method and system for determining physiological characteristics of an animal, it is understood that the disclosure further provides various alternative embodiments. That is, the disclosure can take the form of an entirely hardware embodiment, an entirely software embodiment or an embodiment containing both hardware and software elements. In a preferred embodiment, the disclosure is implemented in software, which includes but is not limited to firmware, resident software, microcode, etc. In one embodiment, the disclosure can take the form of a computer program product accessible from a computer-usable or computer-readable medium providing program code for use by or in connection with a computer or any instruction execution system, which when executed, enables a computer infrastructure to determine physiological characteristics of an animal. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, such as storage system 122, magnetic tape, a removable computer diskette, a random access memory (RAM), a read-only memory (ROM), a tape, a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processing unit 114 coupled directly or indirectly to memory elements through a system bus 118. The memory elements can include local memory, e.g., memory 112, employed during actual execution of the program code, bulk storage (e.g., storage system 122), and cache memories which provide temporary storage of at least some program code in order to reduce the number of times code must be retrieved from bulk storage during execution.

In another embodiment, the disclosure provides a method of generating a system for determining physiological characteristics of an animal. In this case, a computer infrastructure, such as computer infrastructure 102 (FIG. 1), can be obtained (e.g., created, maintained, having made available to, etc.) and one or more systems for performing the process described herein can be obtained (e.g., created, purchased, used, modified, etc.) and deployed to the computer infrastructure. To this extent, the deployment of each system can comprise one or more of: (1) installing program code on a computing device, such as computing device 104 (FIG. 1), from a computer-readable medium; (2) adding one or more computing devices to the computer infrastructure; and (3) incorporating and/or modifying one or more existing systems of the computer infrastructure, to enable the computer infrastructure to perform the process steps of the disclosure.

In still another embodiment, the disclosure provides a business method that performs the process described herein on a subscription, advertising, and/or fee basis. That is, a service provider, such as an application service provider, could offer to determine physiological characteristics of an animal as described herein. In this case, the service provider can manage (e.g., create, maintain, support, etc.) a computer infrastructure, such as computer infrastructure 102 (FIG. 1), that performs the process described herein for one or more customers. In return, the service provider can receive payment from the customer(s) under a subscription and/or fee agreement, receive payment from the sale of advertising to one or more third parties, and/or the like.

As used herein, it is understood that the terms “program code” and “computer program code” are synonymous and mean any expression, in any language, code or notation, of a set of instructions that cause a computing device having an information processing capability to perform a particular function either directly or after any combination of the following: (a) conversion to another language, code or notation; (b) reproduction in a different material form; and/or (c) decompression. To this extent, program code can be embodied as one or more types of program products, such as an application/software program, component software/a library of functions, an operating system, a basic I/O system/driver for a particular computing and/or I/O device, and the like.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A system comprising:
a sensor having an array of electrodes for use in obtaining complex impedance data from a body part of an animal; and
a determinater that compares the complex impedance data with an empirical data model to determine a physiological parameter of the animal, the empirical data model including physiological parameter data versus complex impedance data value correspondence of the animal.

2. The system according to claim 1, wherein the physiological parameter is selected from the group consisting of: osmolarity, lactic acid concentration, ionic concentration and glucose concentration.

3. The system according to claim 2, wherein the ionic concentration is selected from the group consisting of: sodium concentration, chloride concentration, potassium concentration, calcium concentration, bicarbonate concentration, and magnesium concentration.

4. The system according to claim 1, wherein the sensor is formed of a conductive material selected from the group consisting of: silver/silver chloride (Ag/AgCl), platinum and carbon.

5. The system according to claim 1, wherein the array of electrodes comprises a current transmitting electrode, a current sensing electrode, and two voltage sensing electrodes positioned on the body part of the animal in a linear arrangement.

6. The system according to claim 5, wherein the two voltage sensing electrodes are positioned between the current transmitting electrode and the current sensing electrode in the linear arrangement.

7. The system according to claim 5, wherein the current transmitting electrode and the current sensing electrode are positioned between the two voltage sensing electrodes in the linear arrangement.

8. The system according to claim 1, wherein the array of electrodes produce a signal having a frequency range that maximizes extraction of the physiological parameter.

9. The system according to claim 8, wherein the frequency range is between about 100 Hz and about 10 MHz.

10. A method comprising:
obtaining complex impedance data for an animal; and
determining a physiological parameter of the animal based on the complex impedance data.

11. The method according to claim 10, wherein the determining includes using an algorithm.

12. The method according to claim 11, further comprising: generating the algorithm using a multivariate analysis.

13. The method according to claim 12, wherein the multivariate analysis includes using at least one of: pattern recognition, principal component analysis, and structure data analysis.

14. The method according to claim 10, wherein the physiological parameter is selected from the group consisting of: osmolarity, lactic acid concentration, ionic concentration and glucose concentration.

15. The method according to claim 14, wherein the ionic concentration is selected from the group consisting of: sodium concentration, chloride concentration, potassium concentration, calcium concentration, bicarbonate concentration, and magnesium concentration.

16. The method according to claim 10, wherein the determining includes comparing the complex impedance data with an empirical data model, the empirical data model including physiological parameter data versus complex impedance data value correspondence of the animal.

17. The method according to claim 10, wherein the complex impedance data for the animal is obtained from the body part of the animal using a sensor having an array of electrodes, the sensor including a current transmitting electrode, a current sensing electrode, and two voltage sensing electrodes in a linear arrangement.

18. The method according to claim 17, wherein the two voltage sensing electrodes are positioned between the current transmitting electrode and the current sensing electrode in the linear arrangement.

19. The method according to claim 17, wherein the current transmitting electrode and the current sensing electrode are positioned between the two voltage sensing electrodes in the linear arrangement.

20. The method according to claim 17, wherein the sensor is formed of a material selected from the group consisting of: silver/silver chloride (Ag/AgCl), platinum and carbon.

21. The method according to claim 17, wherein the sensor produces a signal having a frequency range that maximizes extraction of the physiological parameter.

22. The method according to claim 21, wherein the frequency range is between about 100 Hz and about 10 MHz.

23. A program product stored on a computer readable medium, which when executed, performs the following:
obtaining complex impedance data for an animal;
determining a physiological parameter of the animal based on the complex impedance data; and
outputting the physiological parameter.

24. The program product according to claim 23, wherein the determining includes comparing the complex impedance data with an empirical data model, the empirical data model including physiological parameter data versus complex impedance data value correspondence of the animal.

25. The program product according to claim 23, wherein the determining includes using an algorithm.

26. The program product according to claim 25, further comprising: generating the algorithm using a multivariate analysis.

27. The program product according to claim 26, wherein the multivariate analysis includes using at least one of: pattern recognition, principal component analysis, and structure data analysis.

28. The program product according to claim 23, wherein the physiological parameter is selected from the group consisting of: osmolarity, lactic acid concentration, ionic concentration and glucose level.

29. The program product according to claim 28, wherein the ionic concentration is selected from the group consisting of: sodium concentration, chloride concentration, potassium concentration, calcium concentration, bicarbonate concentration, and magnesium concentration.

30. The program product according to claim 23, wherein the obtaining includes producing a signal having a frequency range that maximizes extraction of the physiological parameter.

31. The program product according to claim 30, wherein the frequency range is between about 100 Hz and about 10 MHz.

32. A system comprising:
an obtainer for obtaining complex impedance data for an animal; and
a determinater for determining a physiological parameter of the animal based on the complex impedance data.

33. The system according to claim 32, wherein the determinater uses an algorithm to determine the physiological parameter.

34. The system according to claim 32, wherein the determinater includes an empirical data model that includes physiological parameter data versus complex impedance data value correspondence.

35. The system according to claim 32, wherein the physiological parameter is selected from the group consisting of: osmolarity, lactic acid concentration, ionic concentration and glucose level.

36. The system according to claim 35, wherein the ionic concentration is selected from the group consisting of: sodium concentration, chloride concentration, potassium concentration, calcium concentration, bicarbonate concentration, and magnesium concentration.

37. The system according to claim 32, wherein the obtainer obtains complex impedance data for the animal from a sensor.

38. The system according to claim 32, wherein the obtainer obtains complex impedance data for the animal from a data storage device.
